SEQUENCE LISTING (24 sequences)

SEQ ID NO: 1 - protein, 476 aa, Artificial Sequence
  Description of Artificial Sequence Synthetic polypeptide
MTKLILLTGLVLILNLQLGSAYQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYVMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 2 - protein, 476 aa, Artificial Sequence
  Description of Artificial Sequence Synthetic polypeptide
MTKLILLTGLVLILNLQLGSAYQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNAYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 3 - protein, 476 aa, Artificial Sequence
  Description of Artificial Sequence Synthetic polypeptide
MTKLILLTGLVLILNLQLGSAYQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 4 - protein, 476 aa, Artificial Sequence
  Description of Artificial Sequence Synthetic polypeptide
MTKLILLTGLVLILNLQLGSAYQLTCYFTNWAQYRPGLGRFMPDDIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 5 - protein, 476 aa, Artificial Sequence
  Description of Artificial Sequence Synthetic polypeptide
MTKLILLTGLVLILNLQLGSAYQLTCYFTNWAQYRPGLGRFMPDDINPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 6 - protein, 476 aa, Artificial Sequence
  Description of Artificial Sequence Synthetic polypeptide
MTKLILLTGLVLILNLQLGSAYQLTCYFTNWAQYRPGLGRFMPDDINPCLCTHLIYAFAGMQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 7 - protein, 455 aa, Artificial Sequence
  Description of Artificial Sequence Synthetic polypeptide
YQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYVMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 8 - protein, 455 aa, Artificial Sequence
  Description of Artificial Sequence Synthetic polypeptide
YQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNAYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 9 - protein, 455 aa, Artificial Sequence
  Description of Artificial Sequence Synthetic polypeptide
YQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 10 - protein, 455 aa, Artificial Sequence
  Description of Artificial Sequence Synthetic polypeptide
YQLTCYFTNWAQYRPGLGRFMPDDIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 11 - protein, 455 aa, Artificial Sequence
  Description of Artificial Sequence Synthetic polypeptide
YQLTCYFTNWAQYRPGLGRFMPDDINPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 12 - protein, 455 aa, Artificial Sequence
  Description of Artificial Sequence Synthetic polypeptide
YQLTCYFTNWAQYRPGLGRFMPDDINPCLCTHLIYAFAGMQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 13 - protein, 387 aa, Artificial Sequence
  Description of Artificial Sequence Synthetic polypeptide
YQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYVMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAP

SEQ ID NO: 14 - protein, 387 aa, Artificial Sequence
  Description of Artificial Sequence Synthetic polypeptide
YQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNAYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAP

SEQ ID NO: 15 - protein, 387 aa, Artificial Sequence
  Description of Artificial Sequence Synthetic polypeptide
YQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAP

SEQ ID NO: 16 - protein, 387 aa, Artificial Sequence
  Description of Artificial Sequence Synthetic polypeptide
YQLTCYFTNWAQYRPGLGRFMPDDIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAP

SEQ ID NO: 17 - protein, 387 aa, Artificial Sequence
  Description of Artificial Sequence Synthetic polypeptide
YQLTCYFTNWAQYRPGLGRFMPDDINPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAP

SEQ ID NO: 18 - protein, 387 aa, Artificial Sequence
  Description of Artificial Sequence Synthetic polypeptide
YQLTCYFTNWAQYRPGLGRFMPDDINPCLCTHLIYAFAGMQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAP

SEQ ID NO: 19 - protein, 377 aa, Artificial Sequence
  Description of Artificial Sequence Synthetic polypeptide
YQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYVMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPA

SEQ ID NO: 20 - protein, 377 aa, Artificial Sequence
  Description of Artificial Sequence Synthetic polypeptide
YQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNAYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPA

SEQ ID NO: 21 - protein, 377 aa, Artificial Sequence
  Description of Artificial Sequence Synthetic polypeptide
YQLTCYFTNWAQYRPGLGRFMPDNIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPA

SEQ ID NO: 22 - protein, 377 aa, Artificial Sequence
  Description of Artificial Sequence Synthetic polypeptide
YQLTCYFTNWAQYRPGLGRFMPDDIDPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPA

SEQ ID NO: 23 - protein, 377 aa, Artificial Sequence
  Description of Artificial Sequence Synthetic polypeptide
YQLTCYFTNWAQYRPGLGRFMPDDINPCLCTHLIYAFAGRQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPA

SEQ ID NO: 24 - protein, 377 aa, Artificial Sequence
  Description of Artificial Sequence Synthetic polypeptide
YQLTCYFTNWAQYRPGLGRFMPDDINPCLCTHLIYAFAGMQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNTYLNVDYAMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPA

(19) United States
(12) Patent Application Publication
(10) Pub. No.: US 2022/0119790 A1
(43) Pub. Date: Apr. 21, 2022

(54) Mutant Chitinase With Enhanced Expression and Activity

(71) Applicants: The Regents of the University of California, Oakland, CA (US); Washington University, Saint Louis, MO (US)

(72) Inventors: Benjamin Barad, San Diego, CA (US); James Fraser, San Francisco, CA (US); Steven Van Dyken, Saint Louis, MO (US); Richard Locksley, San Francisco, CA (US)

(73) Assignees: The Regents of the University of California, Oakland, CA (US); Washtington University, Saint Louis, MO (US)

(21) Appl. No.: 17/505,561

(22) Filed: Oct. 19, 2021

Related U.S. Application Data
(60) Provisional application No. 63/093,777, filed on Oct. 19, 2020.

(51) Int. Cl. C12N 9/42

(52) U.S. Cl. CPC C12N 9/2442; C12Y 302/01014

(57) ABSTRACT

Provided are engineered novel forms of the acidic mammalian chitinase having enhanced expression and having improved catalytic activity. The novel forms of the mammalian acidic chitinase comprise one or more amino acid substitutions, relative to the native sequence, that impart these improved properties. The novel proteins may be used therapeutically for the prevention and treatment of airway conditions associated with environmental chitin exposure and accumulation.

### CROSS-REFERENCE TO RELATED APPLICATIONS

This application claims the benefit of priority to U.S. Provisional Application Ser. No. 63/093,777 entitled “Mutant Chitinase With Enhanced Expression and Activity,” filed Oct. 19, 2020, the contents which are hereby incorporated by reference.

### STATEMENT REGARDING SEQUENCE LISTING

The instant application contains a Sequence Listing which has been submitted electronically in ASCII format and is hereby incorporated by reference in its entirety. Said ASCII copy, created on Oct. 18, 2021, is named UCSF080NP_SL.txt and is 90,503 bytes in size.

### STATEMENT REGARDING FEDERALLY SPONSORED RESEARCH OR DEVELOPMENT

Not applicable.

### BACKGROUND OF THE INVENTION

Chitin is a polysaccharide made up of β-1,-4-linked N-acetylglucosamine, that is utilized as a major structural element by arthropods and fungi. Chitins are ubiquitous in the environment, comprising various forms of polymers and materials. Previous research suggests that chitin is an environmental insult that initiates innate immune cell activation when aspirated into the lung, for example as described in Van Dyken et al. (2014), “Chitin activates parallel immune modules that direct distinct inflammatory responses via innate lymphoid type 2 and γδ T cells,” Immunity 40, 414-424. Recent work has demonstrated pathological effects of chitin accumulation in the lungs. For example as demonstrated in Van Dyken et al., 2017, Spontaneous Chitin Accumulation in Airways and Age-Related Fibrotic Lung Disease, Cell 169:497-509, environmental chitin spontaneously accumulates in the airway of animals having impaired AMCase activity, resulting in persistent immune activation, and promoting the development of pathological conditions such as fibrosis.

Mammals express two active chitinases, chitotriosidase (Chit1) and acidic mammalian chitinase (AMCase; Chia1), an evolutionarily conserved secreted enzyme that is constitutively expressed in the lung and stomach. Previous work suggests that deficits in chitinase production lead to chronic inflammation and underlies various airway conditions.

Accordingly, it has been proposed to treat chitin-associated conditions by administration of exogenous chitinases to the lung, for example as described in PCT International Patent Application Publication Number WO2018191379, entitled “Chitinase Administration to the Airway to Treat Inflammation and Age-related Pulmonary Fibrosis,” the contents of which are hereby incorporated by reference in their entirety.

Accordingly, there is a need in the art for novel, improved chitinase proteins for use in therapeutic treatments.

### SUMMARY OF THE INVENTION

The inventors of the present disclosure have engineered novel chitinase proteins having enhanced expression in expression vectors such as E. coli and having improved catalytic activity. The scope of the invention encompasses novel forms of the mammalian acidic chitinase comprising one or amino acid substitutions that impart improved properties to the chitinases, referred to herein as “Engineered Chitinases.” Improved properties include, for example, improved expression levels of the protein, improved binding to chitin substrates, and improved catalytic activity.

The scope of the invention further encompasses therapeutic uses of the Engineered Chitinases of the invention for the prevention and treatment of airway conditions associated with chitin accumulation. Conditions that may be treated include asthma, age-related lung fibrosis, inflammation, and other pathological processes and diseases.

The scope of the invention further encompasses various formulations of the Engineered Chitinases of the invention for therapeutic uses, including inhaled forms of the enzyme and devices for delivery of Engineered Chitinases to the airway.

### DETAILED DESCRIPTION OF THE INVENTION

Engineered Chitinases. The scope of the invention encompasses various novel mutant chitinases referred to herein as Engineered Chitinases. The mutants comprise amino acid substitutions in the acidic mammalian chitinase protein sequence, which substitutions surprisingly and unexpectedly impart improved properties to the enzyme. The Engineered Chitinases described herein comprise recombinant proteins produced by molecular biology techniques known in the art.

In a first aspect, the scope of the invention encompasses an engineered human acidic mammalian chitinase having one or both of the following mutations:

A substitution of threonine for alanine at position 239 (A239T); and

A substitution of alanine for valine at position 246 (V246A).

The mutations are described by their position in the human AMCase reference sequence, comprising Uniprot accession number Q9BZP6, as known in the art.

An Engineered Chitinase, as used herein, encompasses a recombinantly produced polypeptide having chitinase activity comprising an acidic mammalian chitinase, variant, or catalytic fragment thereof, wherein the polypeptide comprises the A239T mutation, the V246A mutation, or both the A239T and V246A mutation.

In one embodiment, the Engineered Chitinase is an A239T mutant chitinase comprising a polypeptide having at least 90%, at least 95%, or at least 99% sequence identity to SEQ ID NO: 1. In one embodiment, the Engineered Chitinase is a V246A mutant chitinase comprising a polypeptide having at least 90%, at least 95%, or at least 99% sequence identity to SEQ ID NO: 2. In one embodiment, the Engineered Chitinase is an A239T/V246A double mutant comprising a polypeptide having at least 90%, at least 95%, or at least 99% sequence identity to SEQ ID NO: 3. SEQ ID NO: 1 comprises the full 476 amino acid AMCase sequence having the A239T mutation. SEQ ID NO: 2 comprises the full 476 amino acid AMCase sequence comprising the V246A mutation. SEQ ID NO: 3 comprise the full 476 amino acid AMCase sequence comprising both the A239T and V246A mutations.

The Engineered Chitinases of the invention further encompass variants of the foregoing SEQ ID NO: 1, SEQ ID NO: 2 and SEQ ID NO: 3. A variant, as used herein, encompasses a variation of an enumerated sequence comprising one or more polymorphisms relative to the enumerated sequence, such as amino acid substitutions, amino acid deletions, amino acid additions, truncations of the full sequence, or other variations of the enumerated sequence, wherein the resulting polypeptide retains chitinase activity. In one embodiment, the Engineered Chitinase is a variant of SEQ ID NO: 1 comprising a polypeptide having at least 90% sequence identity, at least 95% sequence identity, or at least 99% sequence identity to SEQ ID NO: 1 or a portion thereof. In one embodiment, the Engineered Chitinase is a variant of SEQ ID NO: 2 comprising a polypeptide having at least 90% sequence identity, at least 95% sequence identity, or at least 99% sequence identity to SEQ ID NO: 2 or a portion thereof. In one embodiment, the Engineered Chitinase is a variant of SEQ ID NO: 3 comprising a polypeptide having at least 90% sequence identity, at least 95% sequence identity, or at least 99% sequence identity to SEQ ID NO: 3 or a portion thereof.

In one embodiment the Engineered Chitinase comprises a catalytic fragment of AMCase, a catalytic fragment comprising a truncation or subsequence of an enumerated sequence which retains chitinase activity and comprises one or both of the A239T mutation and/or V246A mutations. In one embodiment, the truncation is a C-terminal truncation of the AMCase. In one embodiment, the truncation is an N-terminal truncation of the AMCase. In one embodiment, the truncation comprises both a C-terminal and N-terminal truncation of the AMCase.

In some implementations, the Engineered Chitinase of the invention comprises an AMCase comprising one the A239T mutation and/or the V246A mutation wherein the first 21 amino acids of the native AMCase sequence are omitted. The Residues 1-21 of the native AMCase comprise a secretion tag. In some implementations, residues 1-21 of the native AMCase sequence are omitted. In one embodiment, the Engineered Chitinase of the invention comprises an A239T mutant chitinase lacking amino acids 1-21 of the native human AMCase sequence. In one embodiment, the Engineered Chitinase comprises a polypeptide having at least 90%, at least 95% or at least 99% sequence identity to SEQ ID NO: 7. In one embodiment, the Engineered Chitinase of the invention comprises an V246A mutant chitinase lacking amino acids 1-21 of the native human AMCase sequence. In one embodiment, the Engineered Chitinase comprises a polypeptide having at least 90%, at least 95% or at least 99% sequence identity to SEQ ID NO: 8. In one embodiment, the Engineered Chitinase of the invention comprises a A239T and V246A mutant chitinase lacking amino acids 1-21 of the native human AMCase sequence. In one embodiment, the Engineered Chitinase comprises a polypeptide having at least 90%, at least 95% or at least 99% sequence identity to SEQ ID NO: 9.

In some embodiments, the Engineered Chitinase comprises a C-terminal truncation, for example a truncation of amino acids 409-476 or subsequence thereof.

In some embodiments, the Engineered Chitinase comprises an AMCase catalytic fragment comprising substantially the catalytic domain of the enzyme. For example, in one embodiment, the Engineered Chitinase comprises a catalytic fragment comprising amino acids 22-408 of the native human AMCase and further comprises one or both of the A239T and/or V246A mutations. In one embodiment, the Engineered Chitinase is a catalytic fragment comprising a polypeptide having at least 90%, at least 95% or at least 99% sequence identity to SEQ ID NO: 13, comprising amino acids 22-408 of the native human AMCase and further comprising the A239T mutation. In one embodiment, the Engineered Chitinase is a catalytic fragment comprising polypeptide having at least 90%, at least 95% or at least 99% sequence identity to SEQ ID NO: 14, comprising amino acids 22-408 of the native human AMCase and further comprising the V246A mutation. In one embodiment, the Engineered Chitinase is a catalytic fragment comprising polypeptide having at least 90%, at least 95% or at least 99% sequence identity to SEQ ID NO: 15, comprising amino acids 22-408 of the native human AMCase and further comprising both the A239T and V246A mutations.

In one embodiment, the Engineered Chitinase comprises a stabilized catalytic fragment. The stabilized catalytic fragment is a mutant chitinase of the invention comprising a C-terminal truncation of amino acids 399-476 of the native AMCase protein, or a truncation comprising a subsequence thereof, and further comprising the A239T mutant, the V246A mutation, or both the A239T and V246A mutations. The stabilized catalytic fragment is a subsequence of the full mutant AMCase having greater in-vivo stability. For example, in some implementations, the stabilized fragment may comprise amino acids 1-398 or amino acids 22-398 of the native AMCase sequence and will further comprise the A239T and/or V246A mutation. In one embodiment, the Engineered Chitinase comprises is a stabilized catalytic fragment comprising a polypeptide having at least 90%, at least 95% or at least 99% sequence identity to SEQ ID NO: 19, comprising amino acids 22-408 of the native human AMCase and further comprising the A239T mutation. In one embodiment, the Engineered Chitinase is a stabilized catalytic fragment comprising polypeptide having at least 90%, at least 95% or at least 99% sequence identity to SEQ ID NO: 20, comprising amino acids 22-408 of the native human AMCase and further comprising the V246A mutation. In one embodiment, the Engineered Chitinase is a stabilized catalytic fragment comprising polypeptide having at least 90%, at least 95% or at least 99% sequence identity to SEQ ID NO: 21, comprising amino acids 22-408 of the native human AMCase and further comprising both the A239T and V246A mutations.

Additional Mutations. In various implementations, the Engineered Chitinases of the invention comprise additional mutations known to increase the expression, stability, binding affinity, and/or catalytic activity of the chitinase. In one embodiment, the additional mutations include one or more of: N45D, D47N, and R61M, as described in Okawa et al, 2016, “Loss and Gain of Human Acidic Mammalian Chitinase Activity by Nonsynonymous SNPs,” Mol. Biol. Evol. 33(12):3183-3193.

In some embodiments, the Engineered Chitinase of the invention comprises a chitinase having an L364Q mutation, in place of or in addition to the A239T and/or V246A mutations.

In one implementation, the Engineered Chitinase further comprises the N45D mutation. In one embodiment, the Engineered Chitinase comprises an A239T mutation and the D45N mutation. In one embodiment, the Engineered Chitinase comprises the V246 mutation and the D45N mutation. In one embodiment, the Engineered Chitinase comprises the A239T mutation, the V246A mutation, and the D45N mutation.

In one implementation, the Engineered Chitinase further comprises the D47N mutation. In one embodiment, the Engineered Chitinase comprises an A239T mutation and the D47N mutation. In one embodiment, the Engineered Chitinase comprises the V246 mutation and the D47N mutation. In one embodiment, the Engineered Chitinase comprises the A239T mutation, the V246A mutation, and the D47N mutation.

In one implementation, the Engineered Chitinase further comprises the R61M mutation. In one embodiment, the Engineered Chitinase comprises an A239T mutation and the R61M mutation. In one embodiment, the Engineered Chitinase comprises the V246 mutation and the R61M mutation. In one embodiment, the Engineered Chitinase comprises the A239T mutation, the V246A mutation, and the R61M mutation.

In various embodiments combinations of two or more of D45N, N47D, and R61M are combined with A239T, V246A, or both A239T and V246A. In various embodiments, the Engineered Chitinase may comprise a polypeptide having at least 90%, at least 95%, or at least 99% sequence identity to any of SEQ ID NO: 4 (AMCase with A239T and V246A mutations and N45D mutation), SEQ ID NO: 5 (AMCase with A239T and V246A mutation, and N45D and D47N mutations), SEQ ID NO: 6 (AMCase with A239T and V246A mutations and N45D, D47N, and R61M mutations), SEQ ID NO: 10 (amino acids 22-476 of AMCase with both A239T and V246A mutations and with N45D mutation), SEQ ID NO: 11 (amino acids 22-476 of AMCase with both A239T and V246A mutations and with N45D and D47N mutations), SEQ ID NO: 12 (amino acids 22-476 of AMCase with both A239T and V246A mutations and with N45D, D47N, and R61M mutations), SEQ ID NO: 16 (amino acids 22-408 of AMCase with both A239T and V246A mutations and with N45D mutation), SEQ ID NO: 17 (amino acids 22-408 of AMCase with both A239T and V246A mutations and with N45D and D47N mutations), SEQ ID NO: 18 (amino acids 22-408 of AMCase with both A239T and V246A mutations and with N45D, D47N, and R61M mutations), SEQ ID NO: 22 (amino acids 22-398 of AMCase with both A239T and V246A mutations and with N45D mutation), SEQ ID NO: 23 (amino acids 22-398 of AMCase with both A239T and V246A mutations and with N45D and D47N mutations), SEQ ID NO: 24 (amino acids 22-398 of AMCase with both A239T and V246A mutations and with N45D, D47N, and R61M mutations).

Fusion Proteins. To facilitate expression, secretion, purification, or other properties, the Engineered Chitinases of the invention may be expressed as fusion proteins comprising one or more polypeptides that impart functionality, increase expression, facilitate purification, or which improve delivery characteristics of the Engineered Chitinase.

Equivalents. The Engineered Chitinases of the invention further encompasses equivalents of the foregoing disclosed sequences wherein the alanine to threonine substitution of A239T is made at the equivalent position in a different reference sequence and/or the valine to alanine substitution of V246A is made at the equivalent position in a different reference sequence. Alternate reference sequences include, for example, non-human forms of the acidic mammalian chitinase such as a murine, rat, canine, feline, equine, or other non-human AMCase reference sequences.

Nucleic Acid Sequences, Expression Vectors, and Cells. In one aspect, the scope of the invention further encompasses nucleic acid sequences coding for an Engineered Chitinases of the invention. One of skill in the art may readily construct a nucleic acid coding for a selected Engineered Chitinase polypeptide sequence of the invention. Such sequences may be derived from the genetic code, with codon selected for optimized expression in a selected organism or expression system.

In various embodiments, the scope of the invention encompasses a nucleic acid sequence encoding for a polypeptide having at least 90%, at least 95%, or at least 99% sequence identity to a sequence selected from SEQ ID NO: ID 1-24, or a variant thereof. In the nucleic acid sequences of the invention, transcription of the sequence may be placed under the control of any promoter, including, for example, lac, tac, trc, T7, PhoA, Plux, and other promoters known in the art. In various embodiments, the nucleic acid sequence may comprise any of an expression vectors, expression cassettes, a plasmid, transformation vectors, and other nucleic acid constructs known in the art.

The scope of the invention further encompasses organisms or cells engineered to express one or more Engineered Chitinase proteins of the invention, including bacterial, yeast, insect, and mammalian cells and organisms. In one embodiment, the engineered cell comprises an E. coli cell, for example, an E. coli periplasmic expression system. In another embodiment, the nucleic acid sequences of the invention are configured for expression in a cell free expression system.

Therapeutic Formulations of the Engineered Chitinases. The scope of the invention further encompasses formulations and devices for the delivery of an aerosolized Engineered Chitinase of the invention to the target tissues of the subject. Chitinase delivery to the lungs may be accomplished by the use of methods, formulations, and devices known in the art, for example, as described in PCT Patent Application Publication Number WO2002043695, entitled “Stable, aerosolizable suspensions of proteins,” by Cowan; U.S. Pat. No. 5,618,786, entitled “Aerosolization of protein therapeutic agent,” by Roosdorp et al.; and U.S. Pat. No. 9,554,993, entitled “Pulmonary delivery particles comprising an active agent,” by Tarara et al. Formulations may comprise dry powders or may comprise liquid formulations such as suspensions, emulsions, or solutions. Powdered enzymes may be spray dried, lyophilized, or otherwise prepared, and may comprise particulates of an effectively respirable size, for example, in the range of 1 μm to 5 μm.

The scope of the invention encompasses apparatuses for the delivery of the agent to the airway of the subject. Such devices will comprise an apparatus which holds the selected chitinase and which is capable of delivering a controlled dosage of such chitinase to the airway tissues of the subject. The delivery may be accomplished by pumps, vaporizing elements such as heaters or vibrational energy sources, or by the use of compressed gases and propellants, as known in the art. In one embodiment, the device comprises a dry powder inhaler. In one embodiment, the device comprises a metered-dose inhaler. In one embodiment, the device comprises a nebulizer.

Methods of Treatment. The scope of the invention encompasses methods of treating chitin-associated airway conditions by the administration of an Engineered Chitinase. In a general implementation, the method of the invention encompasses:
A method of treating an airway condition in a subject in need of treatment therefor by the administration to the airway of the subject of a therapeutically effective amount of a therapeutic composition comprising an Engineered Chitinase, wherein the Engineered Chitinase comprises the A239T mutant, the V246A mutant, or both the A239T and V246A mutations.

In one embodiment, the method encompasses the administration of an Engineered Chitinase having at least 90%, at least 95%, or at least 99% sequence identity to SEQ ID NO: 1, SEQ ID NO: 7, SEQ ID NO: 13, SEQ ID NO: 19 or a variant thereof to the airway of the subject.

In one embodiment, the method encompasses the administration of an Engineered Chitinase having at least 90%, at least 95%, or at least 99% sequence identity to SEQ ID NO: 2, SEQ ID NO: 8, SEQ ID NO: 14, SEQ ID NO: 20, or a variant thereof to the airway of the subject.

In one embodiment, the method encompasses the administration of an Engineered Chitinase having at least 90%, at least 95%, or at least 99% sequence identity to SEQ ID NO: 3, SEQ ID NO: 9, SEQ ID NO: 15, SEQ ID NO: 21, or a variant thereof to the airway of the subject.

In various embodiments, the subject may be a human subject, such as a subject having or at risk of having a chitin-related condition. In other implementations, the subject may be a non-human animal such as a test animal or veterinary subject. Exemplary non-human subjects include mice, rats, cats, dogs, horses, pigs, and non-human primates. In one embodiment, the subject is an aged subject, for example a subject of at least 40 years of age, at least 45 years of age, at least 50 years of age, at least 55 years of age, or at least 60 years of age.

In various embodiments, the subject is a subject at risk of an airway condition or having an chitin-associated condition. A chitin-associated condition, as used herein, encompasses any condition or state induced, exacerbated, or otherwise mediated by exposure to environmental chitin. Chitin-associated conditions include, for example, including any of: chitinase deficiency, encompassing reduced or insufficient chitinase production; age-related chitinase deficiency; chitinase deficiencies exacerbated or caused by lung dysfunctions or inflammatory conditions; fibrotic lung diseases; COPD; asthma; allergies; and lung or airway inflammation. In one embodiment, the subject may be in need of preventative or prophylactic treatment, for example, being a subject that is exposed to chronic or excessive levels of environmental chitins. In one embodiment, the subject comprises a subject at risk of pulmonary fibrosis due to age.

In other implementations, the method of the invention encompasses the administration of an Engineered Chitinase of the invention to enhance, augment, or replace native chitinase function in the airway or to promote clearance of environmentally derived chitins, for example, accumulations of chitinous species in the lungs.

The Engineered Chitinases are administered in a therapeutically effective amount. A therapeutically effective amount may comprise any amount sufficient to degrade chitins in the airway, for example, chitins in the lungs. One of skill in the art may determine what constitutes a therapeutically effective amount based on the properties of the delivered Engineered Chitinase, including retention time of the delivered agent, the activity of the delivered agent, and other factors known in the art. Exemplary dosages range from 1 ng chitinase to 1 mg chitinase per administration, for example about 10 ng, about 100 ng, about 1 microgram, about 10 micrograms, about 100 micrograms, about 1 mg, or about 10 mg per administration. Administration rate may be varied, exemplary courses include: 10 administrations per day, 5 administrations per day, 1-3 administrations per day, 1 administration every two days, or one administration per week.

### EXAMPLES

To improve the activity of mouse AMCase, error-prone PCR was used to generate libraries of AMCase mutants. Mutants of AMCase were generated via error-prone PCR, then transformed and grown out from individual colonies in 96-well blocks. The recombinant expression approach utilized periplasmic secretion yields enzyme secreted into the media: In 96 well format, the ability of the spent media of individual mutants after protein expression to cleave 4MU-chitobioside was assayed. Comparing these results to both wild-type and engineered catalytically dead mutants, it was found that while most mutations resulted in either total loss of protein activity or similar activity to wild-type, a small number of mutants were much more active than the wild-type. Because these assays were done directly on spent media, the measured activity for each well reports on the combination of the specific activity of the enzyme, expression level, and secretion efficiency.

To determine whether the results represented improvements in activity, the two most active mutants: A239T/L364Q was the most active mutant identified, with a 5-fold improvement in activity, and V246A, which showed a 2-fold improvement in activity were isolated and purified. After purification, the specific activity of the assay was measured using a one-pot continuous-read fluorescent assay based on the previously developed enzyme-coupled assay and replicated the improvements observed in the unpurified screening format. Both mutants improved significantly in k_{cat}, while the A239T/L364Q had a nonsignificant improvement in Kₘ. Structurally, the V246A mutation may have a second-shell interaction stabilizing the active conformation, while L364Q is positioned at the binding site for chitin and may directly improve chitin hydrolysis.

An alternative hypothesis for the increased activity is that the engineered variants have high specificity for the fluorophore or a smaller oligomer. This motivated development of new assays on larger and more complex chitin material. As a first control, the contribution of the catalytic and carbohydrate-binding domain of AMCase was assessed. Due to its small oligomeric size, hydrolysis of the 4MU substrate is likely to be driven only by local interactions in the catalytic domain and the presence of the carbohydrate-binding domain should not affect the reaction rate. In contrast, the carbohydrate-binding domain has been hypothesized to play a role in binding crystalline chitin.

The isolated catalytic domain of AMCase was expressed and purified, as well as the full length enzyme, using an E. coli periplasmic expression approach. The ability of the enzyme to catalyze the breakdown of 4-methylumbelliferone (4MU) conjugated chitobioside was measured using a continuous read approach at pH 7.0. The activities of the two constructs were indistinguishable, either in binding or catalysis.

Next different methods of quantifying hydrolysis of insoluble chitin were tested. Colloidal chitin substrates were used, which are more uniform in size and shape and to have reduced settling times compared to other substrates such as shrimp shell chitin. First it was attempted to measure colloidal chitin hydrolysis by the disappearance of scattering by solid substrate as it is converted into small oligomeric products. A statistically significant difference between the two variants could not be observed with this approach, which was likely limited by the relatively small dynamic range and large amount of enzyme required to produce a measurable change in scattering. Each hydrolysis event only minimally altered the scattering of chitin crystals, and many cuts are likely necessary to solubilize crystals.

Next it was attempted to quantify the production of soluble reducing ends, which were hypothesized to more sensitively report individual catalytic events. The first method used to assay production of soluble reducing ends was a ferricyanide reduction assay: after incubating colloidal chitin with AMCase at 37° C. for up to 18 hr, the reaction was quenched the reaction the nonenzymatic reaction of soluble reducing sugars was quantified with potassium ferricyanide, read out by the disappearance of absorbance at 420 nm. With this assay, it was not able to identify a significant difference in total activity but were able to identify that the inclusion of the carbohydrate-binding domain created a small improvement in Kₘ that was offset by a reduction in the k_{cat} of AMCase. This tradeoff did not result in a large difference in activity. Moreover, the endpoint-based requirements of the assay and of the dynamic range available in measuring reduction in absorbance were limiting. Nest, a new assay was developed based on previous work using chitooligosaccharide oxidase (chitO) in combination with horseradish peroxidase to generate signal specifically from the production of chitin reducing ends. To convert this assay from endpoint to continuous readout, the of fluorogenic substrates for horseradish peroxidase were used, carefully washing the colloidal chitin to enable signal measurement without removal of the insoluble component. This gain-of-signal fluorescent assay had much improved signal-to-noise and sensitivity, and improved quantification of the kinetic parameters of chitinase activity. Using this assay, the tradeoff between improved binding (p=0.02) and loss of maximal catalytic activity (p=0.009) with the inclusion of the carbohydrate-binding domain could be assessed, which resulted in no significant change in total activity.

Broadly, these results demonstrate the value of quantifying chitinase kinetics with bulk substrates with the same care used with model substrates (fluorogenic oligomers). The results suggest that the effectiveness and sensitivity of the one-pot chitooligosaccharide oxidase coupled assay makes it an ideal approach for monitoring chitinase activity. While in some cases, the results of the activity assays closely resembled the 4MU-chitobioside assays, in others, the activities were tremendously different, underscoring the need for quantitative measures of bulk chitin catabolism. This proved to be particularly true for studies of the effects of multiple domains, which necessarily cannot bind the same short oligomer the same way they could a chitin crystal, as well as for engineered variants, in which screening with short fluorogenic substrates led to artifacts that may be related to fluorophore binding. The sensitivity and throughput available with the chitO-coupled assay enabled more precise and quantitative measurements of bulk chitin catabolism than was previously available.

In contrast to the majority of cases, which had reasonable agreement between the bulk experiments and the small oligomers, the foregoing efforts to engineer hyperactive chitinases were limited by the use of the 4MU-chitobioside substrate as a screening tool. The result underscores the need in the future for utilizing frequent counter-screening with bulk chitin when performing selection experiments for chitin processing and matches well with previous results in engineering cellulases, which showed that screening with synthetic substrates had significant pit-falls compared to using insoluble substrates. One challenge to accomplishing this is that, while the chitO assay is more sensitive and high throughput than previous techniques, it is sensitive to free sugars and other components of the media that limits its utility for direct screening. With small scale purification.

With the exception of the engineered mutants, the kinetic parameters measured with the 4MU and bulk chi-tin assays were well aligned, with k_{cat} values that were remarkably similar, suggesting that the 4MU assay effectively captures the chemical step of hydrolysis, and Kₘ values that were on the order of 30 μm for the 4MU-chitobioside and 0.03% w/v for the bulk chitin assay. Under the approximation of infinite polymer length, there is one binding site per N-acetylglucosamine unit. Each chitin monomer unit has a molecular mass of 203.21 g/mol, so 0.03% w/v or 0.3 g/L would correspond to approximately 1.5 mM, 50 times greater than the Kₘ observed for the small oligomeric substrates.

What is claimed is:

1. A recombinant acidic mammalian chitinase comprising an amino acid substitution of threonine for alanine at position 239 of the native acidic mammalian chitinase sequence.

2. The recombinant acidic mammalian chitinase of claim 1, comprising a polypeptide having at least 95% sequence identity to SEQ ID NO: 19.

3. The recombinant acidic mammalian chitinase of claim 2, comprising a polypeptide having at least 95% sequence identity to SEQ ID NO: 13.

4. The recombinant acidic mammalian chitinase of claim 3, comprising a polypeptide having at least 95% sequence identity to SEQ ID NO: 1.

5. The recombinant acidic mammalian chitinase of claim 1, wherein the chitinase further comprises one or more of N45D, D47N, and R61M amino acid substitutions.

6. A recombinant acidic mammalian chitinase comprising an amino acid substitution of alanine for valine at position 246 of the native acidic mammalian chitinase sequence.

7. The recombinant acidic mammalian chitinase of claim 6, comprising a polypeptide having at least 95% sequence identity to SEQ ID NO: 20.

8. The recombinant acidic mammalian chitinase of claim 7, comprising a polypeptide having at least 95% sequence identity to SEQ ID NO: 14.

9. The recombinant acidic mammalian chitinase of claim 8, comprising a polypeptide having at least 95% sequence identity to SEQ ID NO: 2.

10. The recombinant acidic mammalian chitinase of claim 6, wherein the chitinase further comprises one or more of N45D, D47N, and R61M amino acid substitutions.

11. A recombinant acidic mammalian chitinase comprising an amino acid substitution of threonine for alanine at position 239 and an amino acid substitution of alanine for valine at position 246 of the native acidic mammalian chitinase sequence.

12. The recombinant acidic mammalian chitinase of claim 11, comprising a polypeptide having at least 95% sequence identity to SEQ ID NO: 21.

13. The recombinant acidic mammalian chitinase of claim 12, comprising a polypeptide having at least 95% sequence identity to SEQ ID NO: 15.

14. The recombinant acidic mammalian chitinase of claim 13, comprising a polypeptide having at least 95% sequence identity to SEQ ID NO: 3.

15. The recombinant acidic mammalian chitinase of claim 15, wherein the chitinase further comprises one or more of N45D, D47N, and R61M amino acid substitutions.

16. A method of treating a chitin-associated condition in a subject by administration of a therapeutically effective amount of a recombinant acidic mammalian chitinase comprising an amino acid substitution of threonine for alanine at position 239 and/or an amino acid substitution of alanine for valine at position 246 of the native acidic mammalian chitinase sequence.

17. The method of claim 16, wherein the recombinant acidic mammalian chitinase comprises a polypeptide having at least 95% sequence identity to SEQ ID NO: 19, SEQ ID NO: 13, or SEQ ID NO: 1.

18. The method of claim 16, wherein the recombinant acidic mammalian chitinase comprises a polypeptide having at least 95% sequence identity to SEQ ID NO: 20, SEQ ID NO: 14, or SEQ ID NO: 2.

19. The method of claim 16, wherein the recombinant acidic mammalian chitinase comprises a polypeptide having at least 95% sequence identity to SEQ ID NO: 21, SEQ ID NO: 15, or SEQ ID NO: 3.

20. The method of claim 16, wherein the chitin-associated condition is selected from the group consisting of chitinase deficiency; chitinase deficiencies exacerbated or caused by lung dysfunctions or inflammatory conditions; fibrotic lung disease; COPD; asthma; allergies; and lung or airway inflammation.
